(19) United States
(12) Patent Application Publication
(10) Pub. No.: US 2005/0288265 A1
(43) Pub. Date: Dec. 29, 2005

(54) Novel combination of glucocorticoids and pde-4 inhibitors for treating respiratory diseases, allegic diseases, asthma and copd

(76) Inventors: Mathias Locher, Ronneburg (DE); Robert Hermann, Max-Reger-Str (DE)

Correspondence Address: VENABLE LLP, P.O. BOX 34385, WASHINGTON, DC, 20045-9998, US

(21) Appl. No.: 10/523,802

(22) PCT Filed: Aug. 4, 2003
(86) PCT No.: PCT/EP03/08607

§ 371 Date: Feb. 9, 2005

(30) Foreign Application Priority Data
Aug. 6, 2002 (DE) 102-36-688.8

(51) Int. Cl. A61K 31/573; A61K 31/4439; A61K 31/404

(52) U.S. Cl. 514/171; 514/339; 514/408; 514/418

(57) ABSTRACT

The invention relates to a novel combination of a glucocorticoid, especially loteprednol, and at least one phospho-diesterase-4 inhibitor (PDE-4-inhibitor), especially hydroxyindole-derivative N-(3,5-dichloropyridine-4-yl)-2-[1-(4-fluorbenzyl)-5-hydroxyindole-3-yl]-2-oxoacetamide, for a simultaneous, sequential or separate administration in the treatment of respiratory diseases, allergic diseases, asthma and chronic obstructive pulmonary diseases (COPD).

The present invention relates to a novel combination of a glucocorticoid, especially loteprednol, and at least one phosphodiesterase-4 inhibitor (PDE-4 inhibitor), especially the hydroxyindole derivative N-(3,5-di-chloropyridin-4-yl)-2-[1-(4-fluorobenzyl)-5-hydroxyindol-3-yl]-2-oxoacetamide, for a simultaneous, sequential or separate administration in the treatment of respiratory diseases, allergic diseases, asthma and chronic obstructive pulmonary diseases (COPD).

Allergic diseases and chronic obstructive pulmonary diseases (COPD) are based on inflammatory processes characterized by an increased number of inflammatory cells and increased release or secretion of inflammation mediators. Studies over the last 20 years have revealed that inflammation of the respiratory tract is of central importance for the respiratory dysfunction in asthma and COPD. Comparable changes have been observed in allergic inflammations of the nose and of the eyes. Normally, the mucosa is infiltrated by a large number of cells, including mast cells, eosinophils and lymphocytes. These cells release a number of mediators, including in particular interleukin-4 (IL-4), GM-CSF (granulocyte/macrophage colony-stimulating factor) and the tumor necrosis factor α (TNF-α), which eventually bring about the inflammations and the symptoms of allergic diseases and of COPD.

At the present time, a similar anti-inflammatory therapeutic approach is followed for all allergic diseases. The pathology of these diseases has revealed that the inflammatory process in the mucosa of patients primarily determines the symptom activity. Of the anti-inflammatory compounds currently available for the treatment of asthma, rhinitis or conjunctivitis, glucocorticoids are the most effective. Active ingredients which can be administered topically by inhalational, intranasal or intraocular administration are preferably employed. On the basis of the successful use of inhalable glucocorticoids in the treatment and prevention of respiratory inflammations and permanent lung damage in asthma patients, this therapeutic approach has also been applied to COPD patients although there are no data which might unambiguously prove a long-term efficacy of these active ingredients in COPD patients (Whittaker A J, Spiro SG; Curr Opin Pulm Med 2000; 6:104-9).

One of the most important anti-inflammatory properties of glucocorticoids arises from inhibition of cytokine release. It is known that several cytokines such as IL-4, IL-5, GM-CSF and TNF-α are involved in respiratory inflammation. The efficacy of glucocorticoids can in part be explained by the inhibitory effect on cytokine synthesis and cytokine release (Marx et al.; Pulm Pharmacol Ther 2002; 15:7-15).

One disadvantage of glucocorticoids arises from their possible systemic side effects such as, for example, growth retardation or else osteoporosis. Sensible measures for reducing the risk of side effects on topical administration of glucocorticoids include the use of the minimum effective dose or restriction of the systemic availability of the active ingredient. A novel route is opened up by the use of so-called soft steroids. In contrast to other glucocorticoids, most of which undergo degradation to pharmacodynamically inactive metabolites only in the liver, the soft steroids undergo partial metabolic inactivation even at the site of their administration (intranasal, ocular or intrapulmonary). Following this partial local metabolism, only very little, or no, pharmacodynamically active substance reaches the systemic blood circulation, so that the steroid-specific side effects are not to be expected in practice. The most prominent example of this novel class of active ingredients is loteprednol, which is already approved for the therapy of allergic conjunctivitis and uveitis.

A further class of potential therapeutics for allergic diseases and COPD comprises the phosphodiesterase-4 inhibitors. Phosphodiesterase enzymes are responsible for the inactivation of cyclic adenosine monophosphate (cAMP) and cyclic guanosine monophosphate (cGMP). Inhibition of phosphodiesterase-4 leads to an increase in cAMP in the cells, in turn leading to downregulation of the function of virtually all proinflammatory cells or immune cells. It is of interest that inflammatory cells involved in the pathogenesis of diseases such as asthma, conjunctivitis, rhinitis or chronic obstructive pulmonary disease preferentially express the phosphodiesterase-4 enzymes.

In recent years there have been advances in the development of phosphodiesterase-4 inhibitors which can be employed for the therapy of allergic diseases, asthma or COPD. It has been possible to show the in vitro inhibitory activity on cytokine release and the therapeutic efficacy in asthma models for example for the active ingredients roflumilast, cilomilast or else piclamilast (Torphy et al.; Pulm Pharmacol Ther 1999; 12:131-5; Poppe et al.; Allergy 2000; 55(Suppl 63):270; Giembycz M A; Expert Opin Investig Drugs 2001; 10:1361-79; Ezeamuzie C I; Eur J Pharmacol 2001; 417:11-8). There is particular interest in a novel class of substituted hydroxyindoles which are described in DE 19 818 964, DE 19 917 504 and U.S. Pat. No. 6,251,923, and also novel 7-azaindoles which are disclosed in DE 10 053 275 and PCT/EP 01/12376.

It has now surprisingly been found that the novel combination of a glucocorticoid with at least one phosphodiesterase-4 inhibitor is advantageous in the treatment of respiratory diseases, allergic diseases, asthma and/or chronic obstructive pulmonary diseases. Add-on therapy of a phosphodiesterase-4 inhibitor, especially the hydroxyindole derivative N-(3,5-dichloropyridin-4-yl)-2-[1-(4-fluorobenzyl)-5-hydroxyindol-3-yl]-2-oxoacetamide, which can be administered orally, intranasally or by inhalation, with topical glucocorticoids, especially loteprednol, is distinguished by improved therapeutic efficacy as well as by the occurrence of few side effects.

The invention serves to improve the therapy of respiratory diseases, allergic diseases, asthma and chronic obstructive pulmonary diseases, as well as the prophylaxis thereof. It is possible with a phosphodiesterase-4 inhibitor present in the combination and with a glucocorticoid successfully to control the inflammations which underlie the pathological states. Moreover, add-on therapy with phosphodiesterase-4 inhibitor leads to a smaller use of glucocorticoids, thus reducing the risk of side effects.

The present invention therefore relates to a composition which comprises a glucocorticoid and at least one phosphodiesterase-4 inhibitor in fixed or free combination, and to the use thereof for producing a medicament. The invention also relates to a medicament for the treatment of respiratory diseases, allergic diseases, asthma and/or chronic obstructive pulmonary diseases, which comprises as active ingredient a glucocorticoid and at least one phosphodiesterase-4 inhibitor in fixed or free combination, and to a process for the production thereof.

It is possible to employ all glucocorticoids for the purposes of the present invention. So-called soft steroids are preferably used. The examples which may be cited of glucocorticoids which can be employed according to the invention are beclomethasone (9-chloro-11β,17,21-trihydroxy-16β-methyl-1,4-pregnadiene-3,20-dione), especially beclomethasone dipropionate, budesonide (16α,17-butylidenedioxy-11β,21-dihydroxy-1,4-pregnadiene-3,20-dione), ciclesonide (see, for example, WO 98/52542 and literature cited therein), fluticasone (S-(fluoromethyl) 6α,9-difluoro-11β-carbothioate), especially fluticasone propionate, mometasone (9,21-dichloro-11β,17-dihydroxy-16α-methyl-1,4-pregnadiene-3,20-dione), in particular mometasone furoate, and loteprednol, especially loteprednol etabonate (chloromethyl 17α-[(ethoxycarbonyl)oxy]-11β-hydroxy-3-oxoandrosta-1,4-diene-17β-carboxylate).

In a preferred embodiment of the invention, loteprednol and its pharmaceutically acceptable esters, especially loteprednol etabonate, is used as soft steroid. The preparation of loteprednol and loteprednol etabonate is described for example in the German patent DE 3 126 732, the corresponding U.S. Pat. No. 4,996,335 and the corresponding Japanese patent JP-89011037.

Further soft steroids suitable according to the invention are described for example in the German patent DE 3 786 174, the corresponding patent EP 0 334 853 and the corresponding U.S. Pat. No. 4,710,495.

It is possible for the purposes of the present invention to employ all phosphodiesterase-4 inhibitors. These include, in particular but not restrictively, the class of substituted hydroxyindole derivatives which are described in DE 19 818 964, DE 19 917 504 and U.S. Pat. No. 6,251,923, and also novel 7-azaindole derivatives which are disclosed in DE 10 053 275 and PCT/EP 01/12376. Examples of phosphodiesterase-4 inhibitors which can be used according to the invention are rolipram ((R)-4-[3-(cyclopentyloxy)-4-methoxyphenyl]-2-pyrrolidinone), roflumilast (Byk-Gulden), piclamilast (Rhone-Poulenc Rorer), cilomilast (GlaxoSmithKline) and the hydroxyindole derivative N-(3,5-dichloropyridin-4-yl)-2-[1-(4-fluorobenzyl)-5-hydroxyindol-3-yl]-2-oxoacetamide. Particular preference is given to the substituted hydroxyindole derivative N-(3,5-dichloropyridin-4-yl)-2-[1-(4-fluoro-benzyl)-5-hydroxyindol-3-yl]-2-oxoacetamide (“DFHO” hereinafter), which is described for example in DE 19 818 964. The phosphodiesterase-4 inhibitors can also be employed as pharmaceutically acceptable salts as are known to the skilled worker.

The inventive combination of a glucocorticoid, in particular of a soft steroid, with at least one phosphodiesterase-4 inhibitor can be administered both prophylactically and after appearance of symptoms. They can also be used to retard or prevent progression of the diseases.

In a preferred embodiment, a combination of the active ingredients loteprednol etabonate and N-(3,5-dichloropyridin-4-yl)-2-[1-(4-fluorobenzyl)-5-hydroxy-indol-3-yl]-2-oxoacetamide (DFHO) is used.

The following description of experiments serves to explain the inventive teaching in detail without restricting it.

Inhibition of GM-CSF Release from LPS-Stimulated Monocytes

EDTAized human whole blood was mixed with Hanks' buffer in the ratio 1:1. Histopaque 1077 solution (15 ml) was cautiously overlaid with max. 40 ml of the blood: Hanks' mixture and centrifuged (2000 rpm) at room temperature for 30 min. The band enriched with leukocytes was aspirated off, washed twice with Hanks' buffer and transferred into RPMI 1640 medium with Glutamax I (Gibco BRL, Eggenstein). The monocytes were removed through their adherence to the cell culture bottle over a period of two hours. The cells were then thoroughly washed with medium in order to remove non-adherent cells. The resulting monocytes were cultured in RPMI 1640 medium with 10% heat-inactivated fetal calf's serum (FCS) and 100 U/ml penicillin and 100 μg/ml streptomycin in a CO₂ incubator (5% CO₂, 96% relative humidity, 37° C.).

Primary monocytes were seeded in 24-well plates at 5×10⁵ cells/well. The cells were preincubated with the stated test substances for 30 minutes. LPS was then added, and incubation was continued for a period of 24 h. The supernatants were aspirated off and investigated by ELISA.

The amount of secreted human GM-CSF in the cell culture supernatants was determined by using an OptEIA™ human GM-CSF ELISA test (Pharmingen, San Diego). It was carried out in microtiter plates. Anti-human monoclonal antibodies were coupled as antibodies to the plate at 4° C. overnight. This coating and three washes were followed by saturation of nonspecific bindings by means of assay diluent solution™ (PBS with 10% FCS, pH 7.0) (Pharmingen, San Diego) at RT for 1 h. This was followed by incubation with the samples and the standard (recombinant human GM-CSF) at 4° C. overnight. The samples were prepared undiluted or in a dilution of 1:50, of, the standard dilutions according to the protocol starting from a stock solution with 500 pg/ml human GM-CSF. Bound human GM-CSF was detected with the aid of biotinylated monoclonal anti-human GM-CSF antibodies and an avidin-horseradish peroxidase reagent at RT for 1 h. All the steps were followed by washing 5 or 7 times with PBS/0.05% Tween 20. The enzyme activity was determined using substrate solution™ (tetramethylbenzidine (TMB) and hydrogen peroxide, Pharmingen, San Diego) as substrate at RT for 30 min. The enzyme-substrate reaction was stopped with 1M phosphoric acid, and the extinction at 450 nm was measured.

Results

Firstly, dose-activity plots were established separately for N-(3,5-dichloropyridin-4-yl)-2-[1-(4-fluorobenzyl)-5-hydroxyindol-3-yl]-2-oxoacetamide (DFHO) and loteprednol. From these, the IC₅₀ for GM-CSF release from human monocytes was calculated respectively as 3.2 μM for DFHO and 53.7 nM for loteprednol. In further experiments, IC₅₀ values for DFHO and loteprednol were established in the presence of sub-IC₅₀ concentrations of the respective other substance. In these cases, addition of 5 nM DFHO lowered the IC₅₀ for loteprednol from 53.7 nM to 13.4 nM. Conversely, addition of 10 nM loteprednol lowered the IC₅₀ for DFHO from 3.2 μM to 0.06 μM. The IC₅₀ values found for loteprednol for release of TNF and of GM-CSF from LPS-stimulated monocytes correspond to the IC₅₀ values indicated in the literature for other cell systems. This means that the cell system used is valid and suitable, and the investigations which are necessary for the aim of the project with this system come to a reliable conclusion. The IC₅₀ values for DFHO correspond to those values indicated in the patent literature.

When 5 nM DFHO was given, the reduction in the IC₅₀ for loteprednol for TNF release was 65% and for GM-CSF release was 75%. The concentration of 5 nM DFHO is far below the IC₅₀ for this substance, which is respectively 5.7 μM and 3.2 μM, so that no effect is to be observed when 5 nM DFHO is given on its own.

Conversely, the reduction in the IC₅₀ for DFHO for TNF release was 99% and for GM-CSF release was 98% when 10 nM loteprednol was given simultaneously. The concentration of 10 nM loteprednol is far below the IC₅₀ of this substance, which is 85.5 nM and 53.7 nM respectively, so that no effect is to be observed when 10 nM loteprednol is given on its own.

A surprising observation which could not have been predicted by the skilled worker is that there is here a superadditive effect brought about by the simultaneous administration of loteprednol and DFHO on the inhibition of TNF and GM-CSF release.

The dosage forms mentioned below are particularly suitable for administration of the inventive combination of active ingredients.

Thus, the active ingredients present in the combination can for example be administered separately as two oral formulations, or one active ingredient is in the form of an oral formulation and the other is in topical form (intranasal, inhalational).

In one embodiment of the invention, the phosphodiesterase-4 inhibitor can be administered orally. Customary pharmaceutical formulations are used in this case, such as tablets, syrup, capsules, preparations with slowed release (sustained release formulation), pastilles or effervescent granules.

Solid pharmaceutical forms such as tablets may comprise inert ingredients and carriers such as, for example, calcium carbonate, calcium phosphate, sodium phosphate, lactose, starch, mannitol, alginates, gelatin, guar gum, magnesium stearate or aluminum stearate, methyl-cellulose, talc, colloidal silicas, silicone oil, high molecular weight fatty acids (such as stearic acid), agar-agar or vegetable or animal fats and oils, solid high molecular weight polymers (such as polyethylene glycol); preparations suitable for oral administrations may, where appropriate, comprise additional flavorings or sweeteners. The compositions in capsule form can be produced by generally customary processes, for example by using the aforementioned carriers in a hard gelatin capsule shell. For compositions in the form of soft gelatin capsules it is possible to employ pharmaceutical carriers normally used for producing dispersions or suspensions, such as, for example, aqueous gums, celluloses, silicates or oils, which are incorporated into a soft gelatin capsule shell. Syrup formulations normally consist of a suspension or solution of the compound or of a salt thereof in a liquid carrier such as, for example, ethanol, peanut oil, olive oil, glycerol or water, it being possible for flavorings and colorants to be present.

It is possible through topical administration of the inventive combination of active ingredients to achieve therapeutically effective concentrations even with lower dosages. For this reason, topical formulations, which include in particular intranasal and inhalational formulations, are preferred for the purposes of the present invention.

Intranasal preparations may be administered as aqueous or oily solutions, suspensions or emulsions. For the administration of an active ingredient by inhalation, it can be administered in the form of a suspension, solution or emulsion which is present as dry powder or as aerosol, it being possible to use all customary propellants.

In a preferred embodiment of the invention, the phosphodiesterase-4 inhibitor composition is in the form of a nasal spray or of a metered aerosol or of a metered dry powder for inhalation. The glucocorticoid composition is preferably likewise a topical preparation, and for the soft steroid loteprednol a formulation in the form of nasal spray, metered aerosol or metered dry powder for inhalation is again preferred.

The soft steroid loteprednol etabonate employed according to the invention is preferably formulated as suspension in water, with further ingredients such as preservatives, stabilizers, tonicity agents, thickeners, suspension stabilizers, excipients to adjust the pH, buffer systems and wetting agents. For further details of suitable excipients, reference is made for example to DE 19 947 234.

The pharmaceutical preparations of the invention may, besides the glucocorticoid and at least one phosphodiesterase-4 inhibitor active ingredients, comprise further ingredients such as customary preservatives, stabilizers, thickeners, flavorings, etc.

### EXEMPLARY EMBODIMENT

Nasal spray suspension with loteprednol etabonate (1%)

| | | |
|---|---|---|
| Loteprednol etabonate | 1.000 | g |
| Avicel RC 591 | 1.100 | g |
| Polysorbate 80 | 0.100 | g |
| Sorbitol solution 70% | 6.000 | g |
| Sodium edetate | 0.050 | g |
| Benzalkonium chloride | 0.020 | g |
| Purified water | ad 100 | ml |

Production

Introduce 45 kg of purified water into a suitable agitating container with homogenizing device, and homogenize Avicel RC 591 therein at high speed. Then dissolve the substances polysorbate 80, sorbitol solution, sodium edetate and benzalkonium chloride together while agitating.

Subsequently homogenize the active ingredient loteprednol etabonate at high speed until a uniform suspension is produced. Then make up the final volume with purified water and homogenize further. Subsequently evacuate the suspension in order to remove the air bubbles which have been produced. The resulting suspension is subsequently dispensed into bottles which are then provided with a suitable nasal spray pump.

In an advantageous embodiment, the active components of this combination are in the form of a fixed combination, thus simplifying use for the patient. Administration of the active ingredients can in this case take place simultaneously, sequentially or separately in free or fixed combination. They can be administered both in a single-dose form and as two separate formulations, which may be identical or different. Delivery can take place at the same time, simultaneously, or at separate times, by which is meant both short and long intervals such as, for example, administration of loteprednol in the evening and administration of the phosphodiesterase-4 inhibitor in the morning, or vice versa.

The active ingredients can be administered from once to six times a day. The active ingredients are preferably administered once to twice a day, particularly preferably twice a day. The dose of one or more phosphodiesterase-4 inhibitors is approximately from 0.1 to 20 mg per day per adult, preferably between 0.2 and 5 mg. The dose of the glucocorticoid can be in the region of the approved dosage, i.e. in the range from 0.1 to 1.6 mg per day, preferably between 0.2 and 0.8 mg per day. The actual dose depends on the general condition of the patients (age, weight, etc.) and the severity of the disease.

1. A composition comprising a glucocorticoid and at least one phosphodiesterase-4 inhibitor in fixed or free combination.

2. The composition as claimed in claim 1, characterized in that the phosphodiesterase-4 inhibitor is rolipram, piclamilast, roflumilast, cilomilast, the hydroxyindole derivative N-(3,5-dichloropyridin-4-yl)-2-[1-(4-fluorobenzyl)-5-hydroxyindol-3-yl]-2-oxoacetamide (DFHO) or their pharmaceutically acceptable salts or mixtures thereof.

3. The composition as claimed in 1, characterized in that the glucocorticoid is a soft steroid.

4. The composition as claimed in claim 1, characterized in that the glucocorticoid is beclomethasone, budesonide, ciclesonide, fluticasone, mometasone or loteprednol or a pharmaceutically acceptable ester thereof.

5. The composition as claimed in claim 3, characterized in that the glucocorticoid is loteprednol etabonate.

6. A medicament for the treatment of respiratory diseases, allergic diseases, asthma and/or chronic obstructive pulmonary diseases, comprising as active ingredient a glucocorticoid and at least one phosphodiesterase-4 inhibitor in fixed or free combination, where appropriate together with customary excipients or carriers.

7. The medicament as claimed in claim 6, characterized in that it can be administered orally.

8. The medicament as claimed in claim 6, characterized in that it can be administered topically.

9. The medicament as claimed in claim 8, characterized in that it can be administered simultaneously, sequentially or separately from one another, intranasally or by inhalation.

10. The medicament as claimed in claim 8, characterized in that it is an inhalable liquid or solid preparation.

11. The medicament as claimed in claim 6, characterized in that one active ingredient is administered orally and at least one active ingredient is administered topically.

12. The medicament as claimed in claim 6, characterized in that the phosphodiesterase-4 inhibitor(s) can be administered orally.

13. A process for producing a medicament for the treatment and prophylaxis of respiratory diseases, allergic diseases, asthma and/or chronic obstructive pulmonary diseases, comprising as active ingredient a glucocorticoid and at least one phosphodiesterase-4 inhibitor, characterized in that the glucocorticoid and the phosphodiesterase-4 inhibitor(s) are mixed singly or together, where appropriate together with customary excipients and carriers, and the mixture obtained in this way is converted into suitable dosage forms.

14. The use of the fixed or free combination of a glucocorticoid and at least one phosphodiesterase-4 inhibitor for producing a medicament for the treatment and prophylaxis of respiratory diseases, allergic diseases, asthma and/or chronic obstructive pulmonary diseases.

15. The use as claimed in claim 14, characterized in that the glucocorticoid is loteprednol etabonate and the phosphodiesterase-4 inhibitor is the hydroxindole derivative N-(3,5-dichloropyridin-4-yl)-2-[1-(4-fluorobenzyl)-5-hydroxyindol-3-yl]-2-oxoacetamide (DFHO).
